(19) United States
(12) Patent Application Publication
(10) Pub. No.: US 2008/0082083 A1
(43) Pub. Date: Apr. 3, 2008

(54) Perforated expandable implant recovery sheath

(76) Inventors: Sean T. Forde, Watertown, MA (US); Derek F. Wood, Bedford, MA (US); Timothy J. Fallon, Melrose, MA (US)

Correspondence Address: WILMERHALE/BOSTON, 60 STATE STREET, BOSTON, MA, 02109, US

(21) Appl. No.: 11/904,496

(22) Filed: Sep. 27, 2007

Related U.S. Application Data
(60) Provisional application No. 60/847,755, filed on Sep. 28, 2006.

(51) Int. Cl. A61M 25/00

(52) U.S. Cl. 604/527

(57) ABSTRACT

An expandable sheath accommodates a medical device that is being removed from a body with a larger diameter than the sheath. The same sheath may be used to reposition a device, such as an implant for repairing a patent foramen ovale (PFO), within the body to an alternative delivery site. The sheath has one or more perforations on its distal end that permit the distal end portion to expand radially. The sheath may be used to deliver a medical device, surgical instrument, or biological sample.

### CROSS REFERENCE TO RELATED APPLICATIONS

This application claims priority to provisional application Ser. No. 60/847,755, filed Sep. 28, 2006, the entire contents of which is incorporated by reference.

### BACKGROUND

The inventions relate to a sheath or catheter that has an expandable distal end.

In many minimally invasive medical procedures, an introducer sheath or catheter may be placed in a vessel to gain access to a site within a body for a diagnostic or therapeutic procedure. Sheaths and catheters can be used as conduits to pass surgical instruments, implantable devices, or biological matter, such as clots, tissue samples, or other matter. The inner diameter of the sheath is designed as large as possible for the surgical instrument, implant device, or tissue sample to pass through it. It is generally desirable to minimize the outer diameter of the sheath and maximize the inner diameter of the sheath. A small outer diameter is desired to minimize the size of the hole at the insertion site. A smaller outer diameter also provides less disruption to the circulatory pathway. Since the outer diameter may be minimized and the inner diameter may be maximized, the thickness of the wall of the sheath could lack sufficient column strength for insertion into a blood vessel or other circumstances with longitudinally applied forces.

Medical devices that are implanted may require removal from the body or repositioning within the body. The device that is to be removed may be a temporary implant which has performed the desired diagnostic or therapeutic function. Alternatively, a device may be classified as a permanent implant but may require removal for some other reason. Sometimes devices need to be repositioned in the body. One way of repositioning a device is to pull the device back into a catheter (or push the catheter around the device) so that the device is disposed within the catheter. Then the device is repositioned to a desired delivery location and then deployed. The devices that are removed or repositioned may not collapse into a reduced profile configuration easily or completely.

Because the devices may not collapse completely or in a suitable orientation it may be difficult to reconstrain the device in a catheter. Specifically, this difficulty may be compounded by the material that is used to construct the catheter. The catheter walls are optimally designed to be as thin as possible while having sufficient column strength for proper operation. A material commonly selected for the construction of catheters typically has high stiffness or rigidity. The same material properties that are desirable in the construction of the catheter may make the withdrawal of an implant or tissue more difficult because a catheter constructed of a stiff material will not expand to accommodate a device that is being reconstrained after deployment. Also, the distal end of the sheath may also bend back if the implant is pulled against it. This can make it awkward to pass surgical instruments, implantable devices, and tissue samples either in or out of the sheath tip.

### SUMMARY

It is desirable to have a sheath that is suitable for reconstraining large or awkwardly shaped surgical instruments and implantable devices after delivery such that they may be repositioned or removed from the body, including medical devices that are being removed from a body with a larger diameter than that of the sheath. A sheath or sheath constructed according to this description may be used to deliver a medical device, surgical instrument, or biological sample. The same sheath may be used to reposition a device within the body to an alternative delivery site. These sheaths have a reduced risk of splitting or tearing when a device is positioned within the sheath. Although term “sheath” is used in this application, as one skilled in the art would know, term “catheter” could also be used interchangeably.

According to one embodiment, a distal end portion of a sheath is constructed to expand radially and thus facilitate the retrieval and repositioning of surgical tools, implantable devices, or biological matter that have a larger diameter than the unexpanded inner diameter of the sheath. The distal end portion of the sheath may be formed with either a single layer or multiple layers of material which may be the same or different from the materials comprising the rest of the sheath. In one embodiment, the distal end portion of the sheath may have one or more perforations. The perforations extend through the thickness of one or more layers of the sheath. If the device requires removal or repositioning, the perforations in the sheath stretch and expand and the distal end portion of the sheath expands radially if necessary as the device is retrieved into the sheath. Optionally, an elastomeric layer holds the perforated distal end portions of the sheath together and provides an expandable layer to provide column strength of the distal end portion of the sheath and to constrain the device into a low profile. The perforations may extend longitudinally from the distal end to a location up to 15 cm along the length of the sheath or more. Alternatively, the perforations may begin at a location slightly away from the distal end and continue longitudinally for up to 15 cm along the sheath or more. In some embodiments, the holes of the perforation may be round or have an alternative shape, such as oval or slots.

In another embodiment, a perforation proximal to the distal end of the sheath may be combined with a slit placed at the end of the perforation away from the distal end of the sheath. The slit may be a straight line, curved, circular, zig-zag or have some other shapes. This formation keeps the edges of the sheath together, but also permits the greater expansion afforded by a slit. The slit and the perforation could also be reversed. The distal end of the sheath may also have a crowned tip.

The formations described above may be used together and other formations may be used to allow for radial expansion of the sheath as the device is being positioned within the sheath. These formations may or may not require longitudinal contraction. These formations can be present along a portion or the entire length of the distal end portion of the sheath. Other materials can be added to the distal end portion of the sheath, such as wires for strength, coatings to change friction characteristics, and coatings of a different durometer.

The sheath can be an introducer through which surgical instruments and implantable devices such as stents, filters, occluders, or other devices are inserted into a living body. The sheath can also be a retriever through which tissue or other biological matter, surgical instruments, and implantable devices are withdrawn from a living body. The perforations may be aligned with the radial axis or each perforated row may be slanted or curved. The perforations may be formed from a sharp object, such as a knife, or alternative methods may be used to form the perforations.

In another embodiment, the sheath may have a distal end portion that is partially or wholly comprised of braided material. In such a device that uses a braided configuration, the longitudinal length shortens as the radius expands. This embodiment has the advantage that individual segments of the sheath are not separated as the sheath expands radially.

A radially expandable distal end portion of a sheath allows surgical instruments, biological matter, and implantable devices, including such devices as may be folded, compressed, or loaded in the sheath in a specialized manner such that the device can be introduced through a smaller diameter delivery sheath than otherwise possible, to be more easily deployed upon delivery to the desired site within the body. A radially expandable distal end portion of a sheath allows and facilitates retrieval of surgical instruments and implantable devices, including devices that unfold or expand or otherwise deploy in some way after delivery within the body. The expandable distal end portion can accommodate more easily the volume of a partially or wholly deployed device, and can overcome snags resulting from the geometry of a partially or wholly deployed device, reducing trauma to the vessel through which such instruments or implantable devices must be withdrawn. Once a device is retrieved into the sheath, the distal end portion of the sheath can further aid in the complete recovery of a device by acting to compress the device. It is desirable that an expandable distal end portion of a sheath accommodates an article with a larger dimension e.g. diameter than that of the sheath.

These and other features and advantages will become apparent from the drawings and detailed description.

### BRIEF DESCRIPTION OF THE DRAWINGS

The inventions will be more clearly understood by reference to the following detailed description when considered in conjunction with the accompanying drawings, wherein:

FIG. 1 is an overall view of a sheath constructed according to an embodiment;

FIG. 2 is a side perspective view of a distal end of a sheath according to an embodiment of the present invention;

FIG. 3 is a side perspective view of a distal end of a sheath according to an embodiment of the present invention;

FIG. 4 is a side perspective view of a distal end of a sheath according to an embodiment of the present invention;

FIG. 5 is a side perspective view of a distal end of a sheath according to an embodiment of the present invention;

FIG. 6 is a side perspective view of a distal end of a sheath according to an embodiment of the present invention;

FIG. 7 is a side perspective view of distal end of a sheath according to an embodiment of the present invention; and

FIG. 8 is a side perspective view of distal end of a sheath according to an embodiment of the present invention.

### DESCRIPTION OF THE PREFERRED EMBODIMENTS

The present invention provides a sheath that can expand radially at its distal end portion, to accommodate an element (e.g., medical instrument or implantable device) that is larger than the inner diameter of the sheath. At times it is desirable, sometimes necessary, to remove or reposition an implantable device that has been previously deployed. A sheath as described here allows an element to be removed or repositioned by expanding its distal end portion to accommodate the element as the element is brought within the sheath. According to some embodiments, the sheath comprises an elastomeric outer layer, and is configured to reduce the possibility of tearing the elastomeric outer layer longitudinally by the edges of the element being removed or repositioned. For purposes herein, the term “sheath” is used interchangeably with the term “catheter.”

Referring to the drawings, wherein like reference numerals designate identical or corresponding parts throughout the several views, and more particularly to FIG. 1 thereof, a sheath 10 is illustrated with a distal end portion 12. The sheath according to this embodiment is adapted to be introduced into the vasculature in a normal procedure as known to those skilled in the art. The expandable distal end portion 12 can expand radially when an element with a larger diameter than the inner diameter of the catheter is introduced into its distal end. The sheath 10 also includes a hub portion 14 proximal to the distal end portion 12. An element to be inserted into a patient is placed through a proximal end 18 and is intended to exit the sheath 10 at a distal end 20. When the sheath 10 is used to remove or reposition an implantable device, the device enters the sheath 10 at the distal end 20. The element placed, removed or repositioned through the sheath 10 may be an implantable device, including, e.g., stents, filters, occluders, or other devices, or a medical instrument such as a delivery element to deliver an implantable device into a patient's body.

The sheath 10 can be various lengths, such as between 50 cm and 100 cm. The sheath can be longer or shorter as necessary for a particular application. The inner diameter of the catheter 10 is typically between 1.67 mm and 5 mm. According to some embodiments of the invention, the sheath could have a larger or smaller diameter as a particular application warranted. Typical wall thickness of the sheath 10 can vary greatly depending on the material selected and the length of the sheath.

As illustrated in FIG. 1, the distal end portion 12 of the sheath 10 is expandable radially because of a perforation 22 disposed on the distal end portion 12 of the sheath. As used herein, “perforation” refers primarily to a series of small, adjacent holes aligned in a linear fashion. This embodiment incorporates only a single perforation. The perforation 22 allows the distal end portion 12 of the sheath 10 to readily tear and expand radially during a delivery or recovery procedure. According to some embodiments of the invention, the diameter of the holes ranges from about 0.25 mm to about 1.00 mm with a preferred embodiment of about 0.64 mm. In some embodiments, the length of the perforation ranges from about 10 mm to about 25 mm with a preferred embodiment of about 18 mm. In some embodiments, the width of the web between holes ranges from about 0.02 mm to about 0.1 mm with a preferred embodiment of about 0.05 mm. according to one embodiment of the invention, the width of the web between holes are consistent through the perforation. In some embodiments, the perforation starts at the distal end 20 of the sheath 10. In another embodiment, the perforation starts proximal to the distal end 20 of the sheath 10.

U.S. patent application Ser. No. 10/921,484, describes expandable sheath tubing that incorporates slits at the distal end portion of the sheath; this application has the same assignee as the present invention and is incorporated herein by reference. While slits may be suitable for some applications, in others, the use of slits compromises (i.e., reduce) column strength of the distal end portion of the sheath. If an element is not constrained to a sufficiently small profile, i.e., because the slits allow a high degree of expansion and/or the sheath does not have sufficient column strength to constrain the element to a low profile configuration, the element may become caught on the interior part of the sheath where the slits stops and the inner circumference is constricted to the normal inner dimension of the sheath. In one embodiment of this invention, the perforation 22 holds the distal end portion 12 of the sheath 10 together, and thus provides greater column strength at the distal end portion 12, making it less likely to bend, buckle or deform, especially in the longitudinal direction. In a perforated form, the sheath is also able to constrain the element to a low profile configuration, which allows a smooth transition as the element passes from the perforated distal end portion to the non-perforated portion of the sheath. According to some embodiments of the invention, an elastomeric outer layer can be applied to the sheath which will stretch enough to allow the radial expansion of the distal end portion of the sheath as the element is drawn into the sheath and be resilient enough to provide a consistent constraint to the element as it travels through the distal end portion of the sheath.

The perforation 22 is dimensioned, both with respect to the size of the holes and the length of the perforation, to suit the particular application. According to some embodiments of the invention, the webs between the holes of the perforation 22 stretch to allow a radial expansion of the distal end of the sheath to a degree. In other embodiments, the webs between the holes of the perforation 22 tear to allow further radial expansion of the distal end of the sheath. The tearing of the webs between the holes of the perforation 22 is controlled by the size of the webs. Unlike with preformed slits, the stretching and tearing of the webs between the holes of the perforation 22 are limited to the extent necessary to allow adequate radial expansion of the distal end portion of the sheath for recovering purpose. In some instances, the use of perforation 22 also reduces the likelihood that an element will catch on the interior part of the sheath by constraining the element into a smaller profile configuration before it reaches the non perforated portion of the sheath.

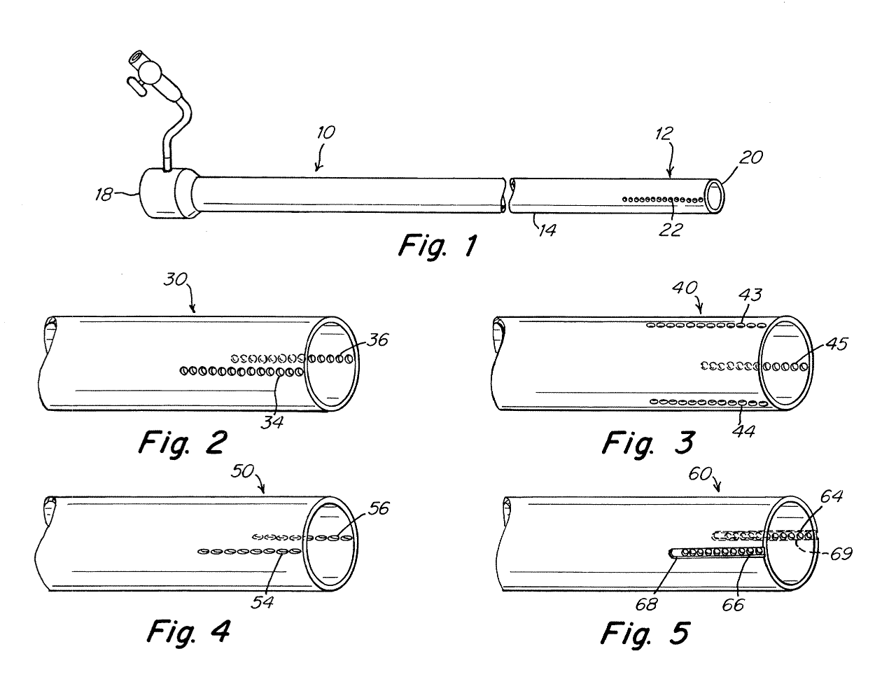

FIG. 2 illustrates an alternative embodiment of the distal end portion 30 of sheath 10. The distal end portion 30 has a first perforation 34 and a second perforation 36. The two perforations 34 and 36 are spaced apart 180 degrees from each other and have the same configurations. In other embodiments, the two perforations could be spaced differently, or could use different numbers of holes or sizes of holes or shapes or have different lengths. Adding additional perforations will generally increase the expandability of the distal end portion of the sheath. An elastomeric member 38, described in detail below, may be disposed of as an outer layer of the perforated sheath. Alternately, or additionally, the elastomeric layer could be on the inside surface of the sheath.

FIG. 3 illustrates an alternative embodiment of the distal end portion 40 of sheath 10. The distal end portion 40 includes three perforations 43, 44, and 45. The three perforations have the same configurations and are equally spaced around the circumference of the distal end portion 40. They can also be spaced at unequal intervals, e.g., at 90 to 180 degrees for three perforations in other embodiments. In other embodiments, the three perforations could be spaced differently, or could use different numbers of holes or sizes of holes or shapes or have different lengths. As described in more detail below, when an element is introduced into the distal end portion of the sheath to be removed or repositioned, the perforations 43, 44 and 45 allow the distal end portion of the sheath to expand further to accommodate the device.

FIG. 4 illustrates an alternative embodiment of the distal end portion 50 of sheath 10. The distal end portion 50 incorporates a first perforation 54 and a second perforation 56. Like the embodiment illustrated in FIG. 2, the perforations 54 and 56 are spaced 180 degrees apart. Unlike the embodiment illustrated in FIG. 2, the shape of the holes of the perforations 54 and 56 are ovoid or slot rather than round. The size of the ovoid or slot can varies according to the application. In a preferred embodiment, the width of the webs between ovoid or slot remains constant. In some embodiments, the width of the web between holes ranges from 0.02 mm to 0.1 mm with a preferred embodiment of 0.05 mm.

FIG. 5 illustrates an alternative embodiment of the distal portion 60 of a sheath 10. The distal end portion 60 incorporates two perforations 64 and 66, spaced 180 degrees apart and having the same configuration, similar to embodiments illustrated in FIG. 2. In this embodiment, a grooved cut 68 and 69 is imposed over each perforation 64 and 66. The grooved cuts 68 and 69 help to further control the radial expansion of the distal end portion 60 of the sheath 10 by controlling the stretching and tearing of the webs between holes of the perforation 64 and 66. By reducing the thickness of the sheath wall at the perforated area, the grooved cuts 68 and 69 help to promote, control and direct stretching and tearing at the particular location of the perforations. The grooved cuts 68 and 69 may be particularly useful in embodiments where the webs between holes of the perforations 64 and 66 are wider.

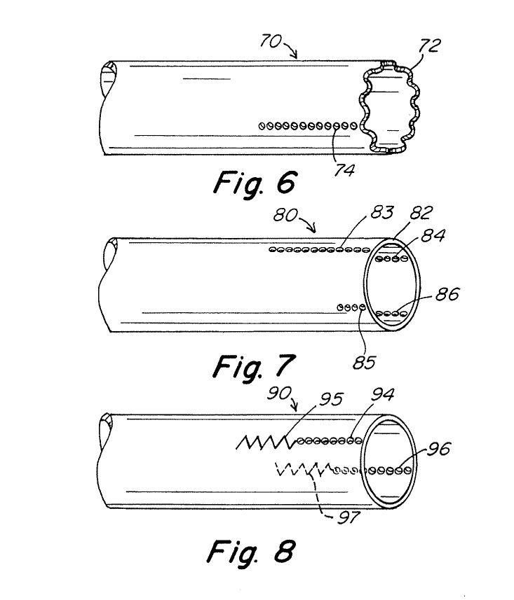

FIG. 6 illustrates an alternate embodiment of the distal end portion 70 for sheath 10. The distal end portion 70 has a crowned distal end 72. The crowned distal end 72 may comprise a plurality of alternating indentations and projection portions. The distal end portion 70 further includes at least a single perforation 74. In one embodiment, the crowned distal end 72 has a ridged profile with concave and projecting wing portions. This profile facilitates the entrance of an element to the distal end 72 of the sheath 10. In some instances, this profile also enables portions of the distal end 72 to bend easily without distorting the entire distal end portion of the sheath if the element is caught on the edge of the distal end 72. While a single perforation 74 is shown in this embodiment, additional perforations of different configurations or slit and perforation combinations may also be used with a crown tip 72. For example, embodiments illustrated in FIGS. 3-5 can also incorporate a crowned distal end 72. In one embodiment, the crown tip may be formed by stretching the distal end of a catheter on a mandrel. In alternative embodiments, the shape of the indentations and projecting portion may be circular, triangular, square, rectangular, or any other suitable shape to facilitate the entrance of an element at the distal end 72 of the sheath 10. In one embodiment, four indentations may be spaced 90 degrees from each other, though other variations in spacing between indentations may be used. In a further alternative embodiment, the distal end of a perforation may intersect or be located proximate to the edge of the indentation or the projecting portion. The distance of the perforation from the crowned distal end 72 may be varied as needed according to the desired application.

FIG. 7 illustrates an alternative embodiment of the distal end portion 80 for sheath 10. The distal end portion 80 incorporates one long perforation 83 and three shorter perforations 84, 85 and 86. This embodiment provides greater column strength than multiple equal length perforations while affording greater radial expansion around the distal end 82 to facilitate easy entrance of the element. The combination of different length perforations, in particular only one single long perforation, allows the expandability of the sheath to be adapted to the particular applications without unduly compromising column strength and stiffness. Multiple short perforations 84, 85, and 86 at the distal end 82 allows the distal end 82 to open up in a funnel shape, reducing the force required for an element to enter the distal end 82. Because only one long perforation 83 is used, the distal end portion 80 has sufficient column strength to withstand typical delivery and recovery procedures.

FIG. 8 illustrates an alternative embodiment of the distal end portion 90 for sheath 10. The distal end portion 90 incorporates both perforations and slits in series. In the illustrated embodiment, a perforation 94 at the distal end portion 90 is followed by a slit 95 that begins where the perforation 94 ends. A second perforation 96, set apart from the first by 180 degrees, is also followed by a second slit 97 (shown in dotted lines). The slit 95 is shown as a zig-zag slit, but could have any other shape adapted to the particular desired application. Alternatively, the positions of the slit 95 and the perforation 94 could be reversed.

In certain embodiments, a perforated sheath may also include an elastomeric layer on the outer distal end portion that provides additional structural integrity. The elastomeric layer may be disposed on the inside surface of the sheath or on the outside surface of the sheath or both. The elastomeric layer is bonded to the sheath layer, such as through heat bonding, adhesives, or other suitable methods. The elastomeric layer could also be affixed to the sheath layer by mechanical means. Although the thickness of the elastomeric layer may vary depending on the needs of a particular application and the material selected, the thickness may be between about 0.025 mm and 0.625 mm, preferably between about 0.050 mm and 0.200 mm. Materials for the elastomeric outer cover may include silicone, polyurethane, or polyether-amide block copolymer. The elastomeric layer(s) allows the distal end portions of the sheath to expand as much as needed to recapture or reposition the element. In some embodiments, the distal end of the elastomeric outer layer is flush with the distal end of the sheath. In other embodiments, the distal end of the elastomeric outer layer extends beyond the distal end of the sheath over a short distance to create an overhang which provides a less stiff and “softer” distal tip to the sheath assembly. This softer distal tip can help to guide an element with structures that could be caught if brought back into contact with a stiffer conduit. This overhang would typically have a length of about 0.125 mm to 12.5 mm and preferably about 2.5 mm, and a thickness of about 0.125 mm to 2.5 mm, and preferably about 0.5 mm to 1.0 mm. In addition to the distal end portion, other sections of the sheath can include multiple layers as shown, for example, in application Ser. No. 10/693,398, which is incorporated herein by reference.

In other embodiments, the expandable distal end portion of the sheath includes a wall formed by braided material. The braid has one or more threads of high-stiffness material knitted or woven together. Braided material has the advantage of readily expanding in the radial direction. This advantage is used to accommodate the introduction of an element into the distal end of the sheath. As the distal end portion of the sheath radially expands to accommodate an element, the braided material contracts longitudinally, i.e. axially. In some embodiments, longitudinal contraction of the distal end portion of the sheath may be achieved by withdrawing an element into the distal end of the sheath. Alternatively, the longitudinal contraction of the distal end portion of the sheath may be produced by the positive action of a control rod or contraction cable. The braided expandable distal end of the catheter may or may not include an elastomeric outer cover.

Features of the embodiments described here include the following: the expandable distal end portion of the sheath facilitates the deployment and retrieval of surgical instruments, implantable devices, and biological matter; use of the expandable distal end portion of the sheath to partially deploy, expand or inflate an implantable device or surgical instrument before delivery of such implantable device or surgical instrument is specifically envisioned. The distal end portion of the sheath radially expands to more easily accommodate implantable device or surgical instrument volumes and overcome any device or instrument geometry that may tear an elastomeric outer layer. The distal end portion of the sheath may or may not be accompanied or enhanced by the addition of other materials such as braids, different tubing, or coatings. The elastomeric outer layer, when present, expands such that the implant will be fully or partially encapsulated within the distal end portion of the sheath. The elastomeric outer layer, when present, also serves to ensure a controlled and consistent expansion of the geometry of distal end portion of the sheath. In addition to the containment of the retrieved device and protection against cut sheath tip areas, the elastomeric material, when present, may extend past the distal end of the sheath layer to form a highly flexible ring that corrects snags, ensuring the successful entry of the device into the distal end of the sheath.

Once the element is retrieved, the sheath continues to aid in the complete recovery by the use of the elastomeric material that can compress the element to ease any remaining size discrepancy between the retrieved element and the nominal diameter of the full length of the sheath. The expandable sheath tip preserves rigidity, column strength, and stiffness where necessary.

In other configurations of sheath, combinations of the above embodiments are possible. For example, one embodiment includes a high-durometer inner wall with a longitudinally-oriented zig-zag slit with perforations, having a cover comprised of a low-durometer braided material. Additionally, the perforations may extend the entire length of the sheath so that an element may be pulled through the length of the sheath. Numerous modifications and variations of the present inventions are possible in light of the above teachings. Although the embodiments have been described in detail for the purpose of illustration, it is understood that such detail is solely for that purpose, and variations can be made by those skilled in the art without departing from the spirit and scope of the inventions.

What is claimed is:

1. A perforated sheath for use with an object within a patient's body comprising an elongated tubular body made of at least one layer of material, the elongated tubular body comprising:
a proximal portion; and
a distal end portion; including a perforation, the perforation having a series of at least one hole extending through the at least one layer of material.

2. The perforated sheath of claim 1, wherein the distal portion of the elongated tubular body comprises a plurality of perforations, wherein each perforation independently comprises at least one hole extending through the at least one layer of material at the distal portion of the elongated tubular body.

3. The perforated sheath of claim 1, wherein the holes are ovoid.

4. The perforated sheath of claim 1, wherein the at least one perforation is located in a longitudinal groove in the elongated tubular body.

5. The perforated sheath of claim 1, wherein the elongated tubular body has a length, and wherein the at least one perforation comprises a plurality of holes extending through the at least one layer of material at the distal end portion of the elongated tubular body, and wherein the plurality of holes extend along the length of the elongated tubular body.

6. The perforated sheath of claim 1, wherein the distal end portion has a distal end having a ridged configuration.

7. The perforated sheath of claim 1, wherein the distal end portion of the elongated tubular body comprises at least two perforations, wherein the first perforation is longer than the second perforation.

8. The perforated sheath of claim 1, wherein the at least one perforation has a distal side and a proximal side,
wherein the elongated tubular body further comprises at least one slit positioned on at least the distal side or the proximal side of the at least one perforation.

9. The perforated sheath of claim 8, wherein the slit is zig-zag, linear, circular, curved, or any combination thereof.

10. The perforated sheath of claim 1, further comprising an elastomeric layer.

11. The perforated sheath of claim 10, wherein the elongated tubular body has an inner surface and an outer surface, and wherein the elastomeric layer is disposed on at least one of the surfaces of the elongated tubular body.

12. The perforated sheath of claim 10, wherein the elastomeric layer has a thickness between about 0.025 and 0.625 mm.

13. The perforated sheath of claim 10, wherein the elastomeric layer has an overhang that extends beyond the distal end portion of the elongated tubular body, and wherein the elastomeric layer is less stiff than the at least one layer of material comprising the elongated tubular body.

14. The perforated sheath of claim 13, wherein the overhang has a length between about 0.125 mm and 12.5 mm.

15. The perforated sheath of claim 10, wherein the elastomeric layer comprises at least one member selected from the group consisting of silicon, polyurethane, polyether-amide block co-polymer.

16. The perforated sheath of claim 1, wherein at least one portion of the elongated tubular body comprises a plurality of layers of material.

17. The perforated sheath of claim 1, wherein the distal end portion of the elongated tubular body comprises a wall formed of a braided material.

18. The perforated sheath of claim 17, wherein the elongated tubular body further comprises an elastomeric layer.

19. The perforated sheath of claim 1, wherein the proximal portion of the elongated tubular body has at least one perforation comprising at least one hole extending through the at least one layer of material.

20. The perforated sheath of claim 19, wherein the at least one perforation on the proximal portion of the elongated tubular body intersects with the least at least one perforation on the distal portion of the elongated tubular body.

21. The perforated sheath of claim 1, wherein at least a portion of the elongated tubular body is covered by a coating.

22. The perforated sheath of claim 1, wherein said coating comprises at least one member selected from the group consisting of an elastomeric layer, tubing, braids, and a drug.

23. A perforated sheath for use with an object within a patient's body comprising a elongated tubular body made of at least one layer of material, the elongated tubular body comprising:
a proximal portion;
a distal end portion; having at least one perforation extending through the at least one layer of material; and
at least one slit positioned on at least a distal side or a proximal side of the at least one perforation.

24. A perforated sheath to deliver or retrieve an object within a patient's body having an elastomeric layer and a elongated tubular body made of at least one layer of material, the elongated tubular body comprises

25. A perforated sheath to deliver or retrieve an object within a patient's body comprising
an elastomeric layer;
a elongated tubular body made of at least one layer of material, wherein the elongated tubular body comprises a proximal portion, and a distal end portion, wherein the distal end portion has at least one perforation comprising at least one hole extending through the at least one layer of material; and
at least one slit positioned on at least a distal side or a proximal side of the at least one perforation.
